(19) United States
(12) Patent Application Publication
(10) Pub. No.: US 2004/0226053 A1
(43) Pub. Date: Nov. 11, 2004

(54) Methods of producing a target molecule in a transgenic animal and purification of the target molecule

(76) Inventors: Harry M. Meade, Newton, MA (US); Scott Fulton, Waltham, MA (US); Yann Echelard, Jamaica Plains, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/863,101

(22) Filed: Jun. 8, 2004

Related U.S. Application Data
(63) Continuation of application No. 09/688,254, filed on Oct. 13, 2000.

(51) Int. Cl. A01K 67/027

(52) U.S. Cl. 800/7; 800/14

(57) ABSTRACT

The invention provides systems and methods for the production and purification of target molecules present in biological systems. The systems and methods according to the invention utilize transgenic expression of multivalent binding polypeptides, as affinity media, to purify such target molecules. The transgenic multivalent binding polypeptides bind both the target molecules, e.g., a bindable epitope of a target molecule, and a matrix.

This application claims the benefit of a previously filed Provisional Application Nos. 60/159,748, filed Oct. 14, 1999, and 60/204,662, filed May 17, 2000, the contents of which are incorporated in their entirety.

### BACKGROUND OF THE INVENTION

Production and purification of polypeptides for therapeutic and diagnostic purposes has become an important industry in recent years. Early attempts at recombinant polypeptide expression utilized plasmid expression vectors in bacteria. For example, U.S. Pat. No. 4,816,397 discloses recombinant expression of immunoglobulins in bacteria. These approaches were limited by the need to break open or lyse the bacteria to obtain the recombinant polypeptide, which was present as part of a complex soup of bacterial proteins and other bacterial substituents. Further limitations resulted from non-native oxidation states for the recombinant polypeptide in the bacteria, which could result in improper polypeptide folding.

Some of these problems were lessened by recombinant expression of polypeptides in mammalian cell culture fermentation systems. U.S. Pat. No. 5,639,640 discloses recombinant expression of human follicle stimulating hormone (FSH) in mammalian cell culture. Unfortunately, such mammalian cell culture fermentation systems are extremely expensive to operate, and the polypeptides thereby produced are part of a complex mixture of proteins present in the growth medium.

Efforts to overcome the problems present in these systems resulted in transgenic expression of recombinant polypeptides in various transgenic animal systems. This effort has allowed the high level transgenic expression of polypeptides, thereby eliminating the need for the expensive mammalian cell culture fermentation systems for production of recombinant polypeptides. However, to be a practical alternative to mammalian cell culture systems, it is important to maintain the health of the animals. Moreover, the polypeptides thereby produced remain a substituent of a complex mixture of milk proteins and methods are need to purify the polypeptides from such mixture.

### SUMMARY OF THE INVENTION

The present invention features methods of producing target molecules in transgenic animals and methods of purifying such target molecules. The target molecule is preferably a target polypeptide. The invention is based, in part, on the discovery that a target polypeptide can be expressed and secreted in an inactive form into the milk of a transgenic animal and then activated. This can allow for avoidance of complications that can arise by producing the protein in active form in the animal. For example, production of certain proteins including enzymes, growth factors, hormones and cytokines, in the milk of a transgenic animal has been found to cause health problems in the animal. Methods of the invention can minimize such problems. The invention also features systems and methods for the purification of these and other target molecules present in biological systems such as milk. These systems and methods can utilize, for example, transgenic expression of multivalent binding polypeptides, such as affinity ligands, into milk to purify such target molecules. Preferably, the transgenic multivalent binding polypeptides can bind both a target molecule and a preselected ligand, e.g., a preselected ligand of a phase separable matrix.

Accordingly, in one aspect, the invention features, a method of producing a target polypeptide in a transgenic animal. The method includes: producing the polypeptide in an inactive state; obtaining the inactive polypeptide; and activating the polypeptide, to thereby provide the target polypeptide.

In a preferred embodiment, the polypeptide is produced in an inactive state in a tissue or product, e.g., a fluid of the transgenic animal, such as milk. In a preferred embodiment, a tissue or product, e.g., milk, containing inactive protein is obtained from the transgenic animal

In one embodiment, the target polypeptide is inactivated by co-expression of the target polypeptide with a binding polypeptide which binds to and inactivates the target polypeptide. In a preferred embodiment, the binding polypeptide and the target polypeptide exist as a complex in the tissue or product, e.g., fluid, e.g., milk.

In a preferred embodiment, the target polypeptide and binding polypeptide are a ligand and counterligand, e.g., the binding polypeptide is a receptor or ligand, or a fragment of either. In another preferred embodiment, the binding polypeptide is an antibody or fragment thereof.

In a preferred embodiment, the nucleic acid encoding the target polypeptide is under the control of a tissue specific promoter, e.g., a promoter which directs expression in mammary epithelial cells. In a preferred embodiment, the nucleic acid encoding the binding polypeptide is under the control of a tissue specific promoter, e.g., a promoter which directs expression in mammary epithelial cells. Preferably, both the nucleic acid encoding the target polypeptide and the nucleic acid encoding the binding polypeptide are under control of the same-type of promoter, e.g., the same type of tissue specific promoter. For example, both can be under the control of a promoter which directs expression in mammary epithelial cells, e.g., both can be under control of the same or a different milk specific promoter. The milk specific promoter can be, e.g., a casein, a lactoglobulin, a lactalbumin or a whey acid protein (WAP) promoter.

In a preferred embodiment, the nucleic acid which encodes the binding polypeptide also encodes a sequence which encodes at least one amino acid exogenous to the binding polypeptide. For example, the nucleic acid can further include an amino acid useful in the purification of the binding polypeptide. In a preferred embodiment, at least one amino acid exogenous to the binding polypeptide can be added, e.g., an added amino acid or amino acids that can bind to a preselected ligand, e.g., a ligand used for purification of the binding polypeptide, e.g., a 6X HIS ligand, a cellulose binding domain (CBD) ligand, a maltose binding protein (MBP) ligand. This amino acid (or amino acids) which can bind to a preselected ligand is also referred to herein as a binding moiety, e.g., a binding moiety capable of binding a preselected ligand or matrix.

In a preferred embodiment, the method further includes separating the inactive polypeptide from the tissue or product, e.g., a fluid, e.g., milk, prior to activation. In a preferred embodiment, the inactive polypeptide is separated from a fluid, e.g., milk, by binding the binding moiety of the binding polypeptide/target polypeptide complex to a preselected ligand, e.g., a 6X HIS ligand (e.g., a metal chelating column), CBD ligand (e.g., cellulose) or a MBP ligand (e.g., maltose).

In yet another embodiment, the target polypeptide is inactivated by modification of a site needed to activate the polypeptide, e.g., modification of a cleavage site. The cleavage site can be, for example, a cleavage site which allows removal of a portion of the polypeptide, e.g., a site which allows for a removal of a pre and/or pro region of the polypeptide. In a preferred embodiment, the cleavage site is modified such that it is no longer recognized by a processing enzyme, e.g., an endogenous processing enzyme which naturally occurs in the tissue or product.

In preferred embodiments, the target polypeptide inactivated by modification of a site, e.g., a cleavage site, can further include an additional site which allows activation, e.g., which allows cleavage such as cleavage of a pre and/or pro-region of a polypeptide. In a preferred embodiment, an endogenous site for proteolytic cleavage can be inactivated and a new site supplied. The new site can be supplied by modifying the endogenous cleavage site and/or by adding an additional amino acid or amino acids. Preferably, the new site is not cleaved by any endogenous processing enzyme which naturally occurs in the tissue or product. For example, a site cleaved by a protease present in the milk of the transgenic animal can be modified, and a site which can be cleaved by contact with an exogenous chemical or enzyme can be added to the polypeptide. In a preferred embodiment, the exogenous chemical is: an acid, e.g., cyanogen bromide. In another preferred embodiment, the exogenous enzyme is an exogenous protease, e.g., chymosin.

In a preferred embodiment, the additional site is cleaved such that the target polypeptide does not include any extraneous sequence. In another preferred embodiment, the site is cleaved such that the target polypeptide includes less than 20, 10, 5, 4, 3, 2, or 1 extraneous amino acid residues.

In a preferred embodiment, the target polypeptide is a human polypeptide. In a preferred embodiment, the polypeptide is: a hormone, a growth factor, a cytokine. In preferred embodiment, the polypeptide is: bone matrix protein (BMP), e.g., BMP-2; erythropoietin; insulin; human growth factor; transforming growth factor-β.

In a preferred embodiment, the transgenic animal is a mammal, e.g., a goat, cow sheep, rabbit, pig, horse, camel, llama, mouse or rat.

In another aspect, the invention features, a method for the transgenic production of a target polypeptide. The method includes: providing a transgenic animal having a nucleic acid which encodes a target polypeptide under the control of a tissue specific promoter, e.g., a promoter which directs expression in mammary epithelial cells, and a nucleic acid which encodes a polypeptide which binds to the target polypeptide and which is under the control of a tissue-specific promoter, e.g., a promoter which directs expression in mammary epithelial cells; allowing the target polypeptide and the binding polypeptide to be expressed in a tissue or product of the transgenic animal, e.g., the milk of the transgenic animal; separating the target polypeptide from the binding polypeptide, to thereby provide the target polypeptide.

In a preferred embodiment, the target polypeptide and binding polypeptide are a ligand and counterligand, e.g., the binding polypeptide is a receptor or ligand, or a fragment of either. In another preferred embodiment, the binding polypeptide is an antibody of fragment thereof.

In a preferred embodiment, the nucleic acid which encodes the binding polypeptide also encodes a sequence which encodes at least one amino acid exogenous to the binding polypeptide. For example, the nucleic acid can further include an amino acid useful in the purification of the binding polypeptide. In a preferred embodiment, at least one amino acid exogenous to the binding polypeptide can be added, e.g., an added amino acid or amino acids that can bind to a preselected ligand, e.g., a ligand used for purification of the binding polypeptide, e.g., a 6X HIS ligand, a cellulose binding domain (CBD) ligand, a maltose binding protein (MBP) ligand. This amino acid (or amino acids) which can bind to a preselected ligand is also referred to herein as a binding moiety, e.g., a binding moiety capable of binding a preselected ligand or matrix.

In a preferred embodiment, the binding polypeptide and target polypeptide exists as a complex in the tissue, for example, the milk of the transgenic animal.

In a preferred embodiment, the method further includes separating the inactive target polypeptide from the tissue or product, e.g., a fluid, e.g., milk, prior to activation. In a preferred embodiment, the inactive polypeptide is separated from a fluid, e.g., milk, by binding the binding moiety of the binding polypeptide/target polypeptide complex to a preselected ligand, e.g., a 6X HIS ligand (e.g., a metal chelating column), CBD ligand (e.g., cellulose) or a MBP ligand (e.g., maltose).

In a preferred embodiment, the promoter which directs expression in mammary epithelial cells is: a casein promoter, e.g., a beta casein promoter; a lactoglobulin promoter, e.g., a beta lactoglobin promoter; a whey acid protein promoter; a lactoalbumin promoter.

In a preferred embodiment, the binding polypeptide and the target polypeptide are under the control of the same-type of tissue specific promoter, e.g., both are under the control of a promoter which directs expression in mammary epithelial cells, e.g., both are under the control of the same or different milk specific promoters.

In a preferred embodiment, the transgenic animal is a non-human mammal. In a preferred embodiment, the mammal is: a goat; a cow; a sheep; a rabbit; a pig; a horse; a llama; a camel; a mouse; a rat.

In another aspect, the invention features a transgenic system for obtaining, e.g., purifying, a target polypeptide. The system includes an animal, e.g., a mammal, which expresses a transgenic multivalent binding polypeptide. The multivalent binding polypeptide includes a first binding moiety which binds the target polypeptide and a second binding moiety which binds a preselected ligand, e.g., a preselected ligand of a matrix.

In a preferred embodiment, the transgenic mammal expresses a transgenic multivalent binding polypeptide in a tissue-specific manner, e.g., the transgenic mammal expresses the multivalent binding polypeptide in a tissue or product, e.g., a fluid (e.g., milk, urine or blood). In a preferred embodiment, the mammal expresses the transgenic multivalent polypeptide in milk and the nucleic acid encoding the multivalent binding polypeptide is under control of a promoter which directs expression in mammary epithelial cells, e.g., a casein, a lactoglobulin, a lactalbumin or a whey acid protein (WAP) promoter.

In a preferred embodiment, the transgenic mammal is a goat, cow, sheep, rabbit, pig, horse, camel, llama, mouse or rat.

In a preferred embodiment, the multivalent binding polypeptide is expressed at high levels in the product, e.g., the fluid, e.g., at least 0.1, 1, 5, 10 mg/ml.

In a preferred embodiment, the system further includes a matrix to which the second binding moiety of the multivalent binding polypeptide can bind.

In a preferred embodiment, the target polypeptide includes a bindable epitope, e.g., an epitope which is bound by the first binding moiety of the multivalent binding polypeptide. In a preferred embodiment, the bindable epitope is removable, e.g., the bindable epitope can be removed, e.g., cleaved, from the remainder of the target polypeptide. In a preferred embodiment, the bindable epitope can be removed by a catalytic moiety. Preferably, the catalytic moiety is part of a second multivalent binding polypeptide, e.g., a second transgenically produced multivalent binding polypeptide. In a preferred embodiment, the second multivalent binding polypeptide, e.g., the second transgenically produced multivalent binding polypeptide, includes a first catalytic domain and a second binding moiety which specifically binds a preselected ligand, e.g., a preselected ligand of a matrix, e.g., a matrix which is different than the matrix specifically bound by the second binding moiety of the first transgenic multivalent binding polypeptide.

In a preferred embodiment, both the target polypeptide and the multivalent binding polypeptide are expressed transgenically into milk. The target polypeptide and the multivalent polypeptide can be expressed by separate animals or by the same animal. In another preferred embodiment, the target polypeptide and the first and second multivalent polypeptides are transgenically expressed in milk. All of these polypeptides can be expressed by different animals or two or more can be produced by the same animal.

Although the following system refers to contacting a multivalent binding polypeptide to the matrix and then to the target, the opposite order is equally appropriate for all embodiments.

In another aspect, the invention features a method for obtaining, e.g., purifying, a target polypeptide from a biological system. Preferably, the target polypeptide includes a bindable epitope. The method includes: contacting a composition which includes a target polypeptide from a biological system with a transgenically expressed multivalent binding polypeptide to form a reaction mixture, to thereby obtain the target polypeptide from the biological system. Preferably, the multivalent binding polypeptide includes a first binding moiety which binds the target molecule, e.g., binds an epitope of the target molecule, and a second binding moiety which binds to a matrix.

In a preferred embodiment, the method further includes: maintaining the mixture of the composition and the multivalent binding polypeptide such that the first binding moiety of the multivalent binding polypeptide can bind the target polypeptide, e.g., the bindable epitope of the target polypeptide. In a preferred embodiment, the method further includes: contacting the mixture with a matrix to which the second binding moiety of the multivalent binding polypeptide can bind, and allowing the second binding moiety of the multivalent binding polypeptide to bind to the matrix. In a preferred embodiment, the method further includes: removing any unbound components of the mixture, and obtaining, e.g., eluting, the target polypeptide from the mixture to thereby obtain the target polypeptide.

In a preferred embodiment, the target polypeptide includes a bindable epitope, e.g., an epitope which is bound by the first binding moiety of the multivalent binding polypeptide. In a preferred embodiment, the bindable epitope is removable, e.g., the bindable epitope can be removed, e.g., cleaved, from the remainder of the target polypeptide. In a preferred embodiment, the bindable epitope can be removed by a catalytic moiety. Preferably, the catalytic moiety is part of a second multivalent binding polypeptide, e.g., a second transgenically produced multivalent binding polypeptide. In a preferred embodiment, the second multivalent binding polypeptide, e.g., the second transgenically produced multivalent binding polypeptide, includes a first catalytic domain and a second binding moiety which specifically binds a matrix, e.g., a matrix which is different than the matrix specifically bound by the second binding moiety of the first transgenic multivalent binding polypeptide.

In a preferred embodiment, the composition from a biological system can be a product obtained from a transgenic animal, e.g., a transgenic mammal. For example, the product can be a fluid, e.g., milk, urine or blood.

In a preferred embodiment, the composition from the biological system is contacted with sample, e.g., a fluid, e.g., milk, urine or blood, containing the transgenic multivalent binding polypeptide.

Preferably, the milk contains high levels of the transgenic multivalent binding protein, e.g., at least 0.1, 1, 5, 10 mg/ml.

In a preferred embodiment, both the target polypeptide and the multivalent binding polypeptide are expressed transgenically into milk. The target polypeptide and the multivalent polypeptide can be expressed by separate animals or by the same animal. In another preferred embodiment, the target polypeptide and the first and second multivalent polypeptides are transgenically expressed in milk. All of these polypeptides can be expressed by different animals or two or more can be produced by the same animal.

Although the following method refers to contacting a multivalent binding polypeptide to the matrix and then to the target, the opposite order is equally appropriate for all embodiments.

In another aspect, the invention features a multiple animal transgenic system which can be used to express and/or obtain, e.g., purify, a target polypeptide, e.g., a target polypeptide which includes a bindable epitope.

The system includes a first transgenic animal, e.g., transgenic mammal, which expresses a transgenic multivalent binding polypeptide which includes a first binding moiety which binds the target polypeptide, e.g., binds the bindable epitope of the target polypeptide, and a second binding moiety which binds a matrix. Preferably, the first transgenic animal expresses the multivalent binding polypeptide into a product, e.g., a fluid, e.g., milk, blood, urine. In a preferred embodiment, the multivalent binding polypeptide is present at high levels in milk of the first transgenic animal, e.g., at levels of at least 0.1, 1, 5, 10 mg/ml.

In a preferred embodiment, the first transgenic animal is a transgenic mammal. Preferably, the transgenic mammal expresses a transgenic multivalent binding polypeptide in a tissue-specific manner, e.g., the transgenic mammal expresses the multivalent binding polypeptide in a fluid, e.g., milk, urine or blood. In a preferred embodiment, the mammal expresses the transgenic multivalent polypeptide in milk and the DNA sequence encoding the multivalent binding polypeptide is under control of a promoter which directs expression in mammary epithelial cells, e.g., a casein, a lactoglobulin, a lactalbumin or a whey acid protein (WAP) promoter.

The system further includes a second transgenic animal, e.g., transgenic mammal, which expresses a target polypeptide, e.g., a target polypeptide which includes a bindable epitope. Preferably, the second transgenic animal expresses the target polypeptide into a sample, e.g., a fluid, e.g., milk, blood, urine. In a preferred embodiment, the target polypeptide is present at high levels in milk of the second animal, e.g., at levels of at least 0.1, 1, 5, 10 mg/ml.

In a preferred embodiment, the second transgenic animal is a transgenic mammal. Preferably, the second transgenic mammal expresses a target polypeptide in a tissue-specific manner, e.g., the transgenic mammal expresses the target polypeptide in a fluid, e.g., milk, urine or blood. In a preferred embodiment, the mammal expresses the target polypeptide in milk and the DNA sequence encoding the target polypeptide is under control of a promoter which directs expression in mammary epithelial cells, e.g., a casein, a lactoglobulin, a lactalbumin or a whey acid protein (WAP) promoter.

In a preferred embodiment, the system further includes a matrix to which the second binding moiety of the multivalent binding polypeptide can bind.

In a preferred embodiment, the bindable epitope is removable, e.g., the bindable epitope can be removed, e.g., cleaved, from the remainder of the target polypeptide. In a preferred embodiment, the bindable epitope can be removed by a catalytic moiety. Preferably, the catalytic moiety is part of a second multivalent binding polypeptide, e.g., a second transgenically produced multivalent binding polypeptide. In a preferred embodiment, the second multivalent binding polypeptide, e.g., the second transgenically produced multivalent binding polypeptide, includes a first catalytic domain and a second binding moiety which specifically binds a matrix, e.g., a matrix which is different than the matrix specifically bound by the second binding moiety of the first transgenic multivalent binding polypeptide. The second multivalent binding polypeptide can be produced by either: the same transgenic animal which expresses the first multivalent binding polypeptide; the transgenic animal which produces the target polypeptide; or a transgenic animal other than the animal which produces the first multivalent binding polypeptide or the target polypeptide.

Although the following system refers to contacting a multivalent binding polypeptide to the matrix and then to the target, the opposite order is equally appropriate for all embodiments.

In another aspect, the invention provides a method for expressing and/or obtaining a target polypeptide, e.g., a target polypeptide which includes a bindable epitope, from a product, e.g., a fluid, e.g., milk, blood, urine. The method includes: obtaining a product, e.g., a fluid, e.g., milk, which includes a target polypeptide from a transgenic animal; and contacting the product, e.g., a fluid, e.g., milk, which includes the target polypeptide, with a transgenic multivalent binding peptide, to form a reaction mixture. Preferably, the multivalent binding polypeptide includes a first binding moiety which can bind the target polypeptide and a second binding moiety which can bind a preselected ligand, e.g., a matrix. In a preferred embodiment, the method further includes maintaining the mixture of the target polypeptide and the multivalent polypeptide such that the first binding moiety of the multivalent binding polypeptide binds to the target polypeptide, e.g., a bindable epitope of the target polypeptide. In a preferred embodiment, the method further includes: contacting the mixture with a matrix to which the second binding moiety binds, and allowing the second binding moiety of the multivalent binding polypeptide to bind to the matrix. The method can further include removing any unbound components of the mixture, and selectively removing, e.g., eluting, the target polypeptide from the mixture.

In a preferred embodiment, the multivalent binding polypeptide is expressed in the milk of a transgenic animal. In a preferred embodiment, the multivalent polypeptide is expressed in the milk of the same transgenic animal which expresses the target polypeptide or it is expressed in the milk of a different transgenic animal. In a preferred embodiment, the transgenic multivalent binding polypeptide is present at high levels in milk from the animal, e.g., at levels of at least 0.1, 1, 5, 10 mg/ml.

Although the following method refers to contacting a multivalent binding polypeptide to the matrix and then to the target, the opposite order is equally appropriate for all embodiments.

In another aspect, the invention features a transgenic animal system used to express and/or obtain, e.g., purify, a target polypeptide, e.g., a target polypeptide which includes a bindable epitope. The system includes a transgenic animal which expresses in its mammary tissue a transgenic multivalent binding polypeptide. Preferably, the multivalent binding polypeptide includes a first binding moiety which specifically binds the target polypeptide, e.g., binds a bindable epitope of a target polypeptide, and a second binding moiety which binds a matrix. In addition, the transgenic animal expresses in its mammary tissue a target polypeptide, e.g., a target polypeptide which includes a bindable epitope.

In a preferred embodiment, the mammal expresses the transgenic multivalent polypeptide in milk and the DNA sequence encoding the multivalent binding polypeptide is under control of a promoter which directs expression in mammary epithelial cells, e.g., a casein, a lactoglobulin, a lactalbumin or a whey acid protein (WAP) promoter. In a preferred embodiment, the mammal expresses the target polypeptide in milk and the DNA sequence encoding the target polypeptide is under the control of a promoter which directs expression in mammary epithelial cells, e.g., a casein, a lactoglobulin, a lactalbumin or a whey acid protein (WAP) promoter.

In a preferred embodiment, the multivalent binding polypeptide is present at high levels in milk of the transgenic animal, e.g., at levels of at least 0.1, 1, 5, 10 mg/ml. In another preferred embodiment, the target polypeptide is present at high levels in milk of the same transgenic animal, e.g., at levels of at least 0.1, 1, 5, 10 mg/ml. Preferably, both the multivalent binding polypeptide and the target molecule are present at high levels, e.g., each are present at levels of at least 0.1, 1, 5, 10 mg/ml.

In a preferred embodiment, the system further includes a matrix to which the second binding moiety specifically binds.

In a preferred embodiment, the bindable epitope of the target polypeptide is removable, e.g., the bindable epitope can be removed, e.g., cleaved, from the remainder of the target polypeptide. In a preferred embodiment, the bindable epitope can be removed by a catalytic moiety. Preferably, the catalytic moiety is part of a second multivalent binding polypeptide, e.g., a second transgenically produced multivalent binding polypeptide. In a preferred embodiment, the second multivalent binding polypeptide, e.g., the second transgenically produced multivalent binding polypeptide, includes a first catalytic domain and a second binding moiety which specifically binds a matrix, e.g., a matrix which is different than the matrix specifically bound by the second binding moiety of the first transgenic multivalent binding polypeptide. The second multivalent binding polypeptide can be: expressed by the same transgenic animal which expressed the first multivalent binding polypeptide and/or target polypeptide: expressed a transgenic animal other than the transgenic animal which expresses the first multivalent binding polypeptide and/or target polypeptide.

Although the following system refers to contacting a multivalent binding polypeptide to the matrix and then to the target, the opposite order is equally appropriate for all embodiments.

In another aspect, the invention features a method for expressing and/or obtaining, e.g., purifying, a target polypeptide, e.g., a target polypeptide which includes a bindable epitope. The method includes: obtaining milk from a transgenic animal which includes a mixture of a transgenic multivalent binding polypeptide and a target polypeptide, e.g., a target polypeptide which includes a bindable epitope. Preferably, the multivalent binding polypeptide includes a first binding moiety which can bind the target polypeptide, e.g., a bindable epitope on the target polypeptide, and a second binding moiety which can bind a matrix. The method can further include: allowing the first binding moiety of the multivalent binding polypeptide to bind to the target polypeptide, e.g., a bindable epitope of the target polypeptide. In a preferred embodiment, the method can further include: contacting such mixture with a matrix to which the second binding moiety specifically binds, and allowing the second binding moiety of the multivalent binding peptide to bind to the matrix. In a preferred embodiment, the method can further include: removing any unbound components of the mixture, and selectively removing, e.g., eluting, the target polypeptide from the mixture.

In a preferred embodiment, the multivalent binding polypeptide is present at high levels in milk of the transgenic animal, e.g., at levels of at least 0.1, 1, 5, 10 mg/ml. In another preferred embodiment, the target polypeptide is present at high levels in milk of the same transgenic animal, e.g., at levels of at least 0.1, 1, 5, 10 mg/ml. Preferably, both the multivalent binding polypeptide and the target molecule are present at high levels, e.g., each are present at levels of at least 0.1, 1, 5, 10 mg/ml.

Although the following method refers to contacting a multivalent binding polypeptide to the matrix and then to the target, the opposite order is equally appropriate for all embodiments.

These systems and methods provide many advantages over previously existing systems. In particular, the systems and methods according to the invention greatly reduce the time and cost of purifying such biological molecules because they obviate the need to separately purify the affinity ligands and couple them to a functionalized affinity matrix.

For purposes of the invention, the following terms have the meanings set forth in this section, unless otherwise explicitly stated. An “epitope”, or a “bindable epitope” is a three-dimensional molecular shape which is specifically bound by a binding moiety. A “binding moiety” is a polypeptide portion of a transgenic multivalent binding polypeptide which specifically binds an epitope.

A “polypeptide”, “protein” and “peptide” are used interchangeably herein. The polypeptide may be glycosylated or unglycosylated. Such polypeptides may include from about three, four, five, six or more amino acids, and may further include secondary, tertiary or quaternary structures, as well as intermolecular associations with other peptides or other non-peptide molecules. Such intermolecular associations may be through, without limitation, covalent bonding (e.g., through disulfide linkages), or through chelation, electrostatic interactions, hydrophobic interactions, hydrogen bonding, ion-dipole interactions, dipole-dipole interactions, or any combination of the above. A “polypeptide portion” comprises from about 3 to about 100 contiguous and/or noncontiguous amino acids, and can be glycosylated or unglycosylated.

A “peptide-containing epitope” is an epitope comprised at least in part of an immunological determinant from an amino acid residue of a peptide. A “carbohydrate-containing epitope” is an epitope comprised at least in part of an immunological determinant from a carbohydrate portion of a molecule. A “lipid-containing epitope” is an epitope comprised at least in part of an immunological determinant from a lipid portion of a molecule. An “immunological determinant” is a three-dimensional shape which contributes to the overall three-dimensional shape of an epitope.

An “immunological determinant from an amino acid residue” is an immunological determinant in which its three-dimensional shape is contributed by all or part of the side-chain and/or alpha-carbon and/or either or both peptide bonds and/or amino and/or carboxy termini of an amino acid residue.

A “transgenic multivalent binding polypeptide” is a transgenically produced polypeptide which includes a first binding moiety which binds a bindable epitope of a target molecule present in a biological system, and a second binding moiety which binds a matrix.

A “target molecule” is any molecule which is to be purified. Although the term “target polypeptide” is used throughout the above description of the systems and methods, other target molecules described herein can be used in any of the embodiments.

A “biological system”, includes in vitro cell or tissue extracts, eukaryotic or prokaryotic cell cultures, tissue cultures, organ cultures, living plants and animals, and extracts of living plants or animals.

A “matrix” is a material which can be phase separated from a biological system.

“Specifically binds” means forming a covalent or non-covalent association with an affinity of at least 10⁴ M⁻¹, more preferably 10⁶ M⁻¹, most preferably at least 10⁹ M⁻¹, under process binding conditions, e.g., pH 4-9 and ionic strength 50 mM to 1 M, which conditions specifically include the conditions present in milk.

A “removable epitope” is an epitope which can be physically separated from a molecule, preferably by chemical or enzymatic cleavage.

“High levels” of expression means at least about 0.1 mg/ml, preferably at least about 0.5 mg/ml, and most preferably at least about 1 mg/ml.

“Selectively eluting” refers to dissociating the target molecule from the transgenic multivalent binding polypeptide, preferably, without dissociating the transgenic multivalent binding polypeptide from the matrix.

As used herein, a “transgenic animal” is a non-human animal in which one or more, and preferably essentially all, of the cells of the animal contain a heterologous nucleic acid introduced by way of human intervention, such as by transgenic techniques known in the art. The transgene can be introduced into the cell, directly or indirectly by introduction into a precursor of the cell, by way of deliberate genetic manipulation, such as by microinjection or by infection with a recombinant virus.

Mammals are defined herein as all animals, excluding humans, which have mammary glands and produce milk.

As used herein, a “dairy animal” refers to a milk producing animal. In preferred embodiments, the dairy animal produce large volumes of milk and have long lactating periods, e.g., cows or goats.

As used herein, the term “plant” refers to either a whole plant, a plant part, a plant cell, or a group of plant cells. The class of plants which can be used in the method of the invention is generally as broad as the class of higher plants amenable to transformation techniques, including both monocotyledonous and dicotyledonous plants. It includes plants of a variety of ploidy levels, including polyploid, diploid and haploid.

The term “purified” target polypeptide, as used herein, refers to a polypeptide that is substantially free of cellular material when produced by a cell which expresses the target polypeptide. The language “substantially free of cellular material” includes preparations of target polypeptide in which the polypeptide is separated from cellular components of the cells in which it is produced. In one embodiment, the language “substantially free of cellular material” includes preparations of target polypeptide having less than about 30% (by dry weight) of non-target polypeptide (also referred to herein as a “protein impurity” or “contaminating protein”), more preferably less than about 20% of non-target polypeptide, still more preferably less than about 10% of non-target polypeptide, and most preferably less than about 5% non-target polypeptide.

Other features and advantages of the invention will be apparent from the following detailed description, and from the claims.

### BRIEF DESCRIPTION OF THE DRAWINGS

FIG. 1 depicts an embodiment where the target molecule and the multivalent binding polypeptide are from separate sources.

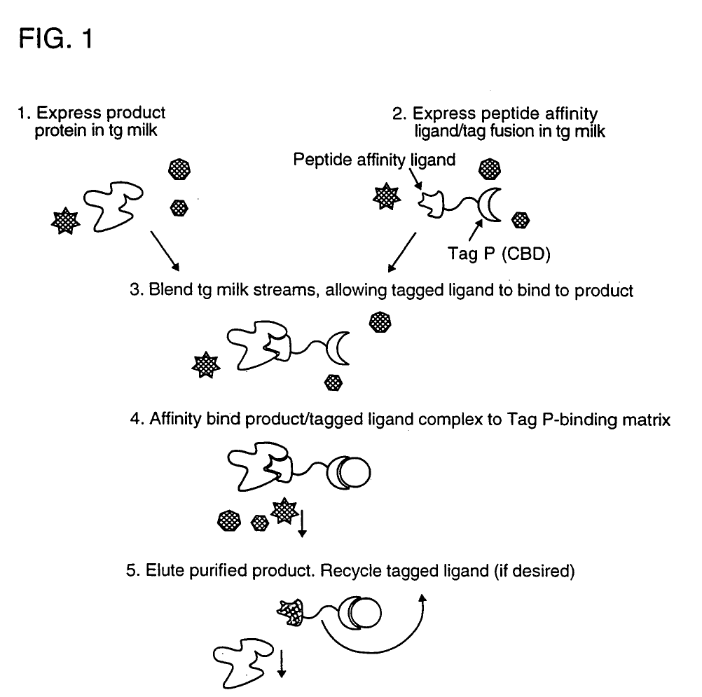

FIG. 2 depicts an embodiment where the target molecule and the transgenic multivalent binding polypeptide are from the same source.

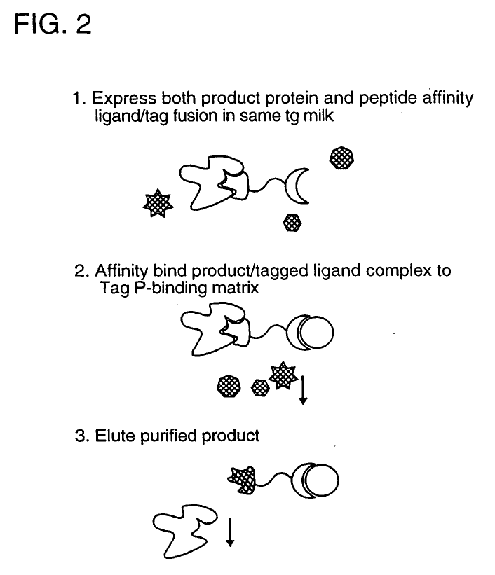

FIG. 3 depicts an embodiment where a removable epitope of the target molecule is removed by a catalytic moiety of a second multivalent binding polypeptide which also binds a matrix. A different matrix is bound by the second multivalent polypeptide than the target molecule/first multivalent binding polypeptide.

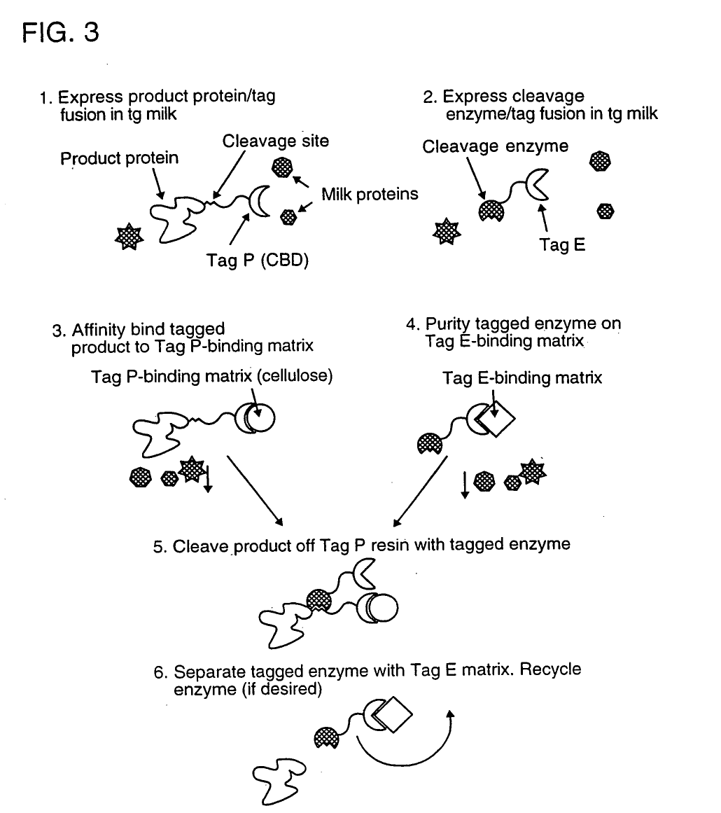

### DETAILED DESCRIPTION OF THE INVENTION

The invention relates to the transgenic expression and purification of polypeptides from biological systems. More particularly, the invention features transgenic systems for expression and purification of polypeptides, and to methods using such systems. The patents and publications cited herein reflect knowledge available to those skilled in the art, and are hereby incorporated by reference in entirety.

The invention provides systems and methods for the purification of target molecules present in biological systems. The systems and methods according to the invention utilize the presence of multivalent binding polypeptides, e.g., transgenically produced multivalent binding polypeptides, as affinity media, to purify such target molecules. Preferably, the multivalent binding polypeptides bind both the target molecule and a matrix, e.g., a phase separable matrix. These systems and methods provide many advantages over previously existing systems. The systems and methods according to the invention greatly reduce the time and cost of purifying such target molecules because they obviate the need to separately purify the affinity ligand and functionalize the affinity matrix. In certain embodiments, the biological molecule to be purified is a polypeptide. In these embodiments, the invention provides systems and methods for both the expression and purification of the target molecules, thereby further simplifying the production process and reducing its cost. Both the target polypeptide and the multivalent binding polypeptide can be expressed transgenically, e.g., into animal milk, either in separate animals or the same animal. Although the following methods and systems generally refer to contacting a multivalent binding polypeptide to the matrix and then to the target, the opposite order is equally appropriate for all embodiments.

The invention provides a transgenic system for purification of a target molecule which includes a bindable epitope. The system can include a transgenic animal which expresses, e.g., in mammary tissue, a transgenic multivalent binding polypeptide. The multivalent polypeptide includes a first binding moiety which binds to a target polypeptide, e.g., binds to a bindable epitope of a target polypeptide, and a second binding moiety which binds a matrix. Preferably, the system further includes a matrix to which the second binding moiety of the multivalent binding polypeptide specifically binds.

The transgenic animal can express the multivalent binding polypeptide in a tissue specific manner. Such tissue specific expression can be obtained by having the sequence encoding the target protein under control of a tissue-specific promoter, e.g. a promoter which directs expression in mammary epithelial cells. Milk specific promoters can include: casein promoters (e.g., α-casein, β-casein, κ-casein or λ-casein promoters); WAP promoters; β-lactoglobin; and lactalbumin.

Target molecules can include: nucleic acids, nucleotides, nucleosides, carbohydrates, lipids, hormones, growth factors, enzyme cofactors, other naturally occurring ligands, and polypeptides. Bindable epitopes of the target molecule can include: carbohydrate-containing epitopes, lipid-containing epitopes and peptide-containing epitopes, as well as epitopes comprising any combination of these. The target molecule can be an endogenous or an exogenous molecule.

The invention can also include a multiple animal transgenic system for expression and/or purification of a target polypeptide, e.g., a target polypeptide having a bindable epitope. The system can include a first transgenic animal which expresses, e.g., in mammary tissue, a transgenic multivalent binding polypeptide. The transgenic multivalent binding polypeptide can include a first binding moiety which specifically binds the target polypeptide, e.g., a bindable epitope of the target polypeptide, and a second binding moiety which specifically binds a matrix. The system can further include a second animal which expresses, e.g., in mammary tissue, a target polypeptide having a bindable epitope. The system can further include a matrix to which the second binding moiety specifically binds.

The second animal can be a transgenic animal which expresses a target polypeptide. For example, the second animal can be a transgenic animal which expresses the target polypeptide in a tissue specific manner, e.g., it expresses the target polypeptide in its mammary tissue. The bindable epitope of a target molecule can be a removable epitope. For example, the epitope can be removed by a catalytic moiety of a second multivalent binding polypeptide, e.g., a second transgenically produced multivalent polypeptide. A second transgenic multivalent binding polypeptide can include a first catalytic domain and a second binding moiety which specifically binds a matrix. Preferably, the matrix specifically bound by the second binding moiety of the second transgenic multivalent binding polypeptide is different than the matrix specifically bound by the second binding moiety of the first transgenic multivalent binding polypeptide. The catalytic domain of a second multivalent binding polypeptide can catalyze the cleavage of the bindable epitope from the removable epitope. For example, the catalytic domain can have an amidase activity.

The invention can also feature a single animal transgenic system for expression and/or purification of a target polypeptide, e.g., a target polypeptide which includes a bindable epitope. The system includes: a transgenic animal which expresses, e.g., in mammary tissue, a transgenic multivalent binding polypeptide and expresses, e.g., in mammary tissue, a target polypeptide. The multivalent binding polypeptide can include a first binding moiety which binds a bindable epitope of the target polypeptide and a second binding moiety which binds a matrix.

The multivalent binding polypeptide and the target polypeptide can be expressed in the same or different tissues. For example, the target polypeptide encoding sequence can be under the control of a general promoter and the multivalent binding polypeptide encoding sequence can be under the control of a tissue specific promoter, or visa versa; the target polypeptide encoding sequence can be under the control of a tissue specific promoter and the multivalent polypeptide encoding sequence can be under the control of a tissue specific promoter for a different tissue-type, e.g., the target polypeptide encoding sequence can be under the control of a urine specific promoter and the multivalent binding polypeptide encoding sequence can be under the control of a milk specific promoter; both the multivalent binding polypeptide encoding sequence and the target polypeptide encoding sequence can be under the control of tissue specific promoters for the same tissue type, e.g., the multivalent binding polypeptide encoding sequence can be under control of a milk specific promoter and the target polypeptide encoding sequence can be under the control of another or the same milk specific promoter.

Preferably, the multivalent binding polypeptide and the target polypeptide are expressed in the milk of transgenic animal. The transgenic multivalent binding polypeptide is, preferably, present at sufficient levels in the milk to bind at least 5%, 10%, 15%, 20%, 30%, 40%, 50%, 60%, 70%, 80%, 85%, 90%, 95%, 98%, 99% or all of the target molecule. For example, the multivalent binding polypeptide can be present at high levels, e.g., at least 0.1, 1, 5, 10 mg/ml, in milk of the transgenic animal. In addition, the target polypeptide is, preferably, present at high levels, e.g., at least 0.1, 1, 5, 10 mg/ml, in milk of the same transgenic animal.

The system can further include a matrix to which the second binding moiety of the multivalent binding polypeptide specifically binds.

The bindable epitope of a target molecule can be a removable epitope. For example, the epitope can be removed by a catalytic moiety of a second multivalent binding polypeptide, e.g., a second transgenically produced multivalent polypeptide. A second transgenic multivalent binding polypeptide can include a first catalytic domain and a second binding moiety which specifically binds a matrix. Preferably, the matrix specifically bound by the second binding moiety of the second transgenic multivalent binding polypeptide is different than the matrix specifically bound by the second binding moiety of the first transgenic multivalent binding polypeptide. The catalytic domain of a second multivalent binding polypeptide can catalyze the cleavage of the bindable epitope from the removable epitope. For example, the catalytic domain can have an amidase activity.

The target polypeptide can be expressed in an inactive form in the animal's mammary tissue. This aspect can be particularly advantageous in situations in which it is not desired to have the target polypeptide perform its biological function in the mammary tissue. For example, the first binding moiety of the multivalent binding polypeptide can bind the target polypeptide in a manner which prevents it from performing its biological function until it is selectively eluted or the bindable epitope can be a removable epitope in which the target polypeptide is not biologically active until the epitope is removed. In some embodiments, the binding moiety can remove a removable epitope.

The invention also features methods for obtaining, e.g., purifying, a target molecule which includes a bindable epitope from a biological system. The method can utilize any of the systems described above. The method can include: contacting a multivalent binding polypeptide, e.g., any multivalent binding polypeptide described herein, with a composition of matter from a biological system to form a reaction mixture. The composition of matter can include a target molecule having a bindable epitope. Within the mixture, the first binding moiety of the multivalent binding polypeptide can bind the bindable epitope of the target molecule. Preferably, the reaction mixture is maintained such that the first binding moiety of the multivalent binding polypeptide binds to the bindable epitope of the target molecule. The mixture can further be contacted with a matrix to which the second binding moiety binds. In the presence of the matrix, the second binding moiety of the multivalent binding peptide can bind the matrix, and any unbound components of the mixture can be removed. The target molecule can then be obtained, e.g., eluted, from the composition of matter.

The method can also include providing a sample, e.g., a fluid, e.g., milk, urine, blood, which includes a target molecule. The sample can be contacted with a multivalent binding polypeptide described herein, to obtain the target molecule. For example, a sample which includes a target polypeptide can be obtained from an animal, e.g., a transgenic animal. Preferably, the sample can be obtained from an animal which expresses the target molecule in its milk, e.g., the sample can be obtained by milking the animal. The animal can be any transgenic animal which includes a nucleotide sequence encoding the target polypeptide under the control of a milk specific promoter.

The invention also features methods of obtaining, e.g., purifying, a target molecule, e.g., a target molecule having a bindable epitope. The method can include: providing a fluid, e.g., milk, which includes a target molecule, e.g., a target polypeptide, and a multivalent binding polypeptide, a multivalent binding polypeptide described herein. For example, the fluid, e.g., milk, can be obtained from a transgenic animal which expresses the target polypeptide and the multivalent binding polypeptide in the fluid, e.g., milk. The method further includes contacting the fluid with a matrix to which the second binding moiety binds. In the presence of the matrix, the second binding moiety of the multivalent binding peptide can bind the matrix, and any unbound components of the mixture can be removed. The target molecule can then be obtained, e.g., eluted, from the composition of matter.

Preferably, the composition of matter from a biological system is contacted with milk containing sufficient levels of a multivalent binding polypeptide to obtain at least 5%, 10%, 20%, 30%, 40%, 50%, 60%, 70%, 80%, 85%, 90%, 95%, 98%, 99% or all of the target molecule from the composition of matter. For example; the milk can contain high levels of the transgenic multivalent binding protein due to its expression in mammary tissue and secretion into the milk. Preferably, the transgenic multivalent binding polypeptide is present in milk at levels of at least 0.1, 1, 5, 10 mg/ml.

Biological systems include, for example, plant, fungi, bacterial and animal products. Compositions from biological systems can include: conditioned media, tissue extracts, organs and bodily fluids (e.g., blood, plasma, serum, sweat, saliva, urine, milk, etc.). Such plant, fungi, bacterial and animal products may include polypeptides expressed from endogenous or exogenously supplied genes.

In any of the methods, the bindable epitope of a target molecule can be a removable epitope. For example, the epitope can be removed by a catalytic moiety of a second multivalent binding polypeptide, e.g., a second transgenically produced multivalent polypeptide. A second transgenic multivalent binding polypeptide can include a first catalytic domain and a second binding moiety which specifically binds a matrix. Preferably, the matrix specifically bound by the second binding moiety of the second transgenic multivalent binding polypeptide is different than the matrix specifically bound by the second binding moiety of the first transgenic multivalent binding polypeptide. The catalytic domain of a second multivalent binding polypeptide can catalyze the cleavage of the bindable epitope from the removable epitope. For example, the catalytic domain can have an amidase activity. When the epitope is a removable epitope, the target polypeptide can be obtained from the mixture by removing the epitope from the target molecule.

The target polypeptide can be expressed in an inactive form in the animal's mammary tissue. This aspect can be particularly advantageous in situations in which it is not desired to have the target polypeptide perform its biological function in the mammary tissue. For example, the first binding moiety of the multivalent binding polypeptide can bind the target polypeptide in a manner which prevents it from performing its biological function until it is selectively eluted or the bindable epitope can be a removable epitope in which the target polypeptide is not biologically active until the epitope is removed. In some embodiments, the binding moiety can remove a removable epitope.

Binding Moiety of Multivalent Binding Polypeptides Which Bind a Target Molecule

The multivalent binding polypeptides of the invention include a first binding moiety which can bind to a target molecule, e.g., a bindable epitope of a target molecule. Preferred first binding moieties include: an antibody, an Fab or F(ab)₂ fragment thereof, or a polypeptide portion comprising a complementarity determining region of an antibody which specifically binds a bindable epitope of the target molecule, a ligand or receptor which binds a bindable epitope of a target molecule, or any other polypeptide portion which specifically binds a bindable epitope of the target molecule, e.g., a polypeptide selected for binding in, e.g., a phage display or 2 hybrid assay.

Antibodies or Fragments Thereof

It is well known in the art how to make antibodies and various antibody derivatives. For example, Jones et al., Nature 321: 522-525 (1986) discloses replacing the CDRs of a human antibody with those from a mouse antibody. Marx, Science 229: 455-456 (1985) discusses chimeric antibodies having mouse variable regions and human constant regions. Rodwell, Nature 342: 99-100 (1989) discusses lower molecular weight recognition elements derived from antibody CDR information. Clackson, Br. J. Rheumatol. 3052: 36-39 (1991) discusses genetically engineered monoclonal antibodies, including Fv fragment derivatives, single chain antibodies, fusion proteins chimeric antibodies and humanized rodent antibodies. Reichman et al., Nature 332: 323-327 (1988) discloses a human antibody on which rat hypervariable regions have been grafted. Verhoeyen, et al., Science 239: 1534-1536 (1988) teaches grafting of a mouse antigen binding site onto a human antibody.

Binding Moiety of Multivalent Binding Polypeptides Which Bind a Matrix

The multivalent binding polypeptides of the invention also include a second binding moiety which binds to a matrix. Second binding moieties can include, for example, antibodies or antibody derivatives, in which case the matrix comprises the antigen or epitope thereof which is specifically bound by the antibody. In such embodiments, the interaction between the antibody and antigen must be sufficiently avid to prevent dissociation under conditions which would elute the target molecule. Other preferred second-binding moiety-matrix pairs include, without limitation, polyhistidine-nickel metal chelate (elution with <250 mM imidazole or low pH), streptavidin-biotin (elution with 6 M urea, pH 4.0), Flag™ peptide-specific MAb (elution with pH 3.0 or 2-5 mM EDTA), S-peptide-S-protein ribonuclease (elution after self-cleavage), glutathione-S-transferase-glutathione (elution with 5-10 mM reduced glutathione), protein A or synthetic ZZ domain of protein A-IgG (elution at low pH), IgG Fc region-protein A (elution at low pH), maltose-binding domains-cross-linked amylose (elution with 10 mM maltose) and cellulose binding domains (CBD)-cellulose or chitin (elution with water or >4 M guanidinium or 1 M or greater NaOH. The cellulose binding domains-cellulose or chitin pair is presently most preferred. For purification of antibodies, a most preferred embodiment comprises a first binding moiety from protein L and a second binding moiety from CBD.

Target Molecules

The target molecule can be an endogenous molecule, e.g., an endogenous polypeptide or an exogenous molecule, e.g., polypeptide. For example, the exogenous polypeptide can be a polypeptide which is naturally expressed in the animal but not in a particular tissue or at lower levels in the tissue which it is being expressed, or the exogenous polypeptide can be a polypeptide which is not expressed in the animal, e.g., the animal is a non-human animal which expresses a human polypeptide. Preferably, the target molecule includes a bindable epitope.

The bindable epitope can be a removable epitope. The removable epitope can be removed after the selective elution of the target molecule. Such removable epitopes can be derived from polypeptide portions of proteins which are edited after translation by self-splicing, e.g., the so-called “intein”-containing proteins. Alternatively, the removable epitope can be joined to the remainder of the target molecule by a specific protease recognition and cleavage site, provided however that no other such site in the target molecule is accessible to the protease. Preferably, the protease can be attached to the matrix. In some embodiments, the transgenic multivalent binding polypeptide itself can incorporate the protease activity, either as part of the first binding moiety, or as a separate, flexibly-attached additional binding moiety. When the protease activity is part of the first binding moiety, it is preferred that it have the ability to bind the bindable epitope under one set of conditions and cleave the bindable epitope under a second, different set of conditions.

Transgenic Mammals

Methods for generating non-human transgenic mammals are known in the art. Such methods can involve introducing DNA constructs into the germ line of a mammal to make a transgenic mammal. For example, one or several copies of the construct may be incorporated into the genome of a mammalian embryo by standard transgenic techniques.

Although bovines and goats are preferred, other non-human mammals can be used. Preferred non-human mammals are ruminants, e.g., cows, sheep, camels or goats. Additional examples of preferred non-human animals include oxen, horses, llamas, pigs, mice and rats. For nuclear transfer techniques, the mammal used as the source of cells, e.g., genetically engineered cell, will depend on the transgenic mammal to be obtained. By way of an example, the genome from a bovine should be used from nuclear transfer with a bovine oocyte.

Methods for the preparation of a variety of transgenic animals are known in the art. Protocols for producing transgenic goats are known in the art. For example, a transgene can be introduced into the germline of a goat by microinjection as described, for example, in Ebert et al. (1994) Bio/Technology 12:699, or nuclear transfer techniques as described, for example, in PCT Application WO 98/30683. A protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. A protocol for the production of a transgenic rat can be found in Bader and Ganten, Clinical and Experimental Pharmacology and Physiology, Supp. 3:S81-S87, 1996. A protocol for the production of a transgenic cow can be found in U.S. Pat. No. 5,741,957, PCT Application WO 98/30683, and Transgenic Animal Technology, A Handbook, 1994, ed., Carl A. Pinkert, Academic Press, Inc. A protocol for the production of a transgenic sheep can be found in PCT Publication WO 97/07669, and Transgenic Animal Technology, A Handbook, 1994, ed., Carl A. Pinkert, Academic Press, Inc.

Transfected Cell Lines

Genetically engineered cells for production of a transgenic mammal by nuclear transfer can be obtained from a cell line into which a nucleic acid of interest, e.g., a nucleic acid which encodes a protein, has been introduced.

A construct can be introduced into a cell via conventional transformation or transfection techniques. As used herein, the terms “transfection” and “transformation” include a variety of techniques for introducing a transgenic sequence into a host cell, including calcium phosphate or calcium chloride co-precipitation, DEAE-dextrane-mediated transfection, lipofection, or electroporation. In addition, biological vectors, e.g., viral vectors can be used as described below. Suitable methods for transforming or transfecting host cells can be found in Sambrook et al., Molecular Cloning: A Laboratory Manuel, 2^{nd} ed., Cold Spring Harbor Laboratory, (Cold Spring Harbor Laboratory Press, Cold Spring Harbor, N.Y., 1989), and other suitable laboratory manuals.

Two useful approaches are electroporation and lipofection. Brief examples of each are described below.

The DNA construct can be stably introduced into a donor cell line, e.g., an embryonic cell, e.g., an embryonic somatic cell line, by electroporation using the following protocol: the cells are resuspended in PBS at about 4×10⁶ cells/ml. Fifty micorgrams of linearized DNA is added to the 0.5 ml cell suspension, and the suspension is placed in a 0.4 cm electrode gap cuvette (Biorad). Electroporation is performed using a Biorad Gene Pulser electroporator with a 330 volt pulse at 25 mA, 1000 microFarad and infinite resistance. If the DNA construct contains a Neomyocin resistance gene for selection, neomyocin resistant clones are selected following incubation with 350 microgram/ml of G418 (GibcoBRL) for 15 days.

The DNA construct can be stably introduced into a donor cell line by lipofection using a protocol such as the following: about 2×10⁵ cells are plated into a 3.5 cmiameter well and transfected with 2 micrograms of linearized DNA using LipfectAMINE™ (GibcoBRL). Forty-eight hours after transfection, the cells are split 1:1000 and 1:5000 and, if the DNA construct contains a neomyosin resistance gene for selection, G418 is added to a final concentration of 0.35 mg/ml. Neomyocin resistant clones are isolated and expanded for cyropreservation as well as nuclear transfer.

Tissue-Specific Expression of Proteins

It is often desirable to express a protein, e.g., a heterologous protein, in a specific tissue or fluid, e.g., the milk, blood or urine, of a transgenic animal. The heterologous protein can be recovered from the tissue or fluid in which it is expressed. For example, it is often desirable to express the heterologous protein in milk. Methods for producing a heterologous protein under the control of a milk specific promoter are described below. In addition, other tissue-specific promoters, as well as, other regulatory elements, e.g., signal sequences and sequence which enhance secretion of non-secreted proteins, are described below.

Milk Specific Promoters

Useful transcriptional promoters are those promoters that are preferentially activated in mammary epithelial cells, including promoters that control the genes encoding milk proteins such as caseins, beta lactoglobulin (Clark et al., (1989) Bio/Technology 7: 487-492), whey acid protein (Gordon et al. (1987) Bio/Technology 5: 1183-1187), and lactalbumin (Soulier et al., (1992) FEBS Letts. 297: 13). Casein promoters may be derived from the alpha, beta, gamma or kappa casein genes of any mammalian species; a preferred promoter is derived from the goat beta casein gene (DiTullio, (1992) Bio/Technology 10:74-77). Milk-specific protein promoter or the promoters that are specifically activated in mammary tissue can be derived from cDNA or genomic sequences. Preferably, they are genomic in origin.

DNA sequence information is available for the mammary gland specific genes listed above, in at least one, and often in several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985) (rat β-casein); Yu-Lee & Rosen, J. Biol. Chem. 258, 10794-10804 (1983) (rat γ-casein); Hall, Biochem. J. 242, 735-742 (1987) (α-lactalbumin human); Stewart, Nucleic Acids Res. 12, 389 (1984) (bovine αs1 and κ casein cDNAs); Gorodetsky et al., Gene 66, 87-96 (1988) (bovine β casein); Alexander et al., Eur. J. Biochem. 178, 395-401 (1988) (bovine κ casein); Brignon et al., FEBS Lett. 188, 48-55 (1977) (bovine αS2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine β lactoglobulin); Vilotte et al., Biochimie 69, 609-620 (1987) (bovine α-lactalbumin). The structure and function of the various milk protein genes are reviewed by Mercier & Vilotte, J. Dairy Sci. 76, 3079-3098 (1993) (incorporated by reference in its entirety for all purposes). If additional flanking sequence are useful in optimizing expression of the heterologous protein, such sequences can be cloned using the existing sequences as probes. Mammary-gland specific regulatory sequences from different organisms can be obtained by screening libraries from such organisms using known cognate nucleotide sequences, or antibodies to cognate proteins as probes.

Signal Sequences

Useful signal sequences are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the construct, which are described below. The size of the signal sequence is not critical. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin can be used. A preferred signal sequence is the goat β-casein signal sequence.

Signal sequences from other secreted proteins, e.g., proteins secreted by kidney cells, pancreatic cells or liver cells, can also be used. Preferably, the signal sequence results in the secretion of proteins into, for example, urine or blood.

Other Tissue-Specific Promoters

Other tissue-specific promoters which provide expression in a particular tissue can be used. Tissue specific promoters are promoters which are expressed more strongly in a particular tissue than in others. Tissue specific promoters are often expressed essentially exclusively in the specific tissue. For example, if the altered protein is normally expressed in the liver, a liver-specific promoter can be used. This will be the case when a suppressor tRNA is used to alter serum albumin. In this situation, a transgenic sequence encoding the suppressor tRNA can be under the control of a liver-specific promoter.

Tissue-specific promoters which can be used include: a neural-specific promoter, e.g., nestin, Wnt-1, Pax-1, Engrailed-1, Engrailed-2, Sonic hedgehog; a liver-specific promoter, e.g., albumin, alpha-1 antirypsin; a muscle-specific promoter, e.g., myogenin, actin, MyoD, myosin; an oocyte specific promoter, e.g., ZP1, ZP2, ZP3; a testes-specific promoter, e.g., protamin, fertilin, synaptonemal complex protein-1; a blood-specific promoter, e.g., globulin, GATA-1, porphobilinogen deaminase; a lung-specific promoter, e.g., surfactant protein C; a skin- or wool-specific promoter, e.g., keratin, elastin; endothelium-specific promoters, e.g., Tie-1, Tie-2; and a bone-specific promoter, e.g., BMP.

In addition, general promoters can be used for expression in several tissues. Examples of general promoters include β-actin, ROSA-21, PGK, FOS, c-myc, Jun-A, and Jun-B.

Insulator Sequences

The DNA constructs used to make a transgenic animal can include at least one insulator sequence. The terms “insulator”, “insulator sequence” and “insulator element” are used interchangeably herein. An insulator element is a control element which insulates the transcription of genes placed within its range of action but which does not perturb gene expression, either negatively or positively. Preferably, an insulator sequence is inserted on either side of the DNA sequence to be transcribed. For example, the insulator can be positioned about 200 bp to about 1 kb, 5′ from the promoter, and at least about 1 kb to 5 kb from the promoter, at the 3′ end of the gene of interest. The distance of the insulator sequence from the promoter and the 3′ end of the gene of interest can be determined by those skilled in the art, depending on the relative sizes of the gene of interest, the promoter and the enhancer used in the construct. In addition, more than one insulator sequence can be positioned 5′ from the promoter or at the 3′ end of the transgene. For example, two or more insulator sequences can be positioned 5′ from the promoter. The insulator or insulators at the 3′ end of the transgene can be positioned at the 3′ end of the gene of interest, or at the 3′ end of a 3′ regulatory sequence, e.g., a 3′ untranslated region (UTR) or a 3′ flanking sequence.

A preferred insulator is a DNA segment which encompasses the 5′ end of the chicken β-globin locus and corresponds to the chicken 5′ constitutive hypersensitive site as described in PCT Publication 94/23046, the contents of which is incorporated herein by reference.

DNA Constructs

A cassette which encodes a heterologous protein can be assembled as a construct which includes a promoter, e.g., a promoter for a specific tissue, e.g., for mammary epithelial cells, e.g., a casein promoter, e.g., a goat beta casein promoter, a milk-specific signal sequence, e.g., a casein signal sequence, e.g., a β-casein signal sequence, and a DNA encoding the heterologous protein.

The construct can also include a 3′ untranslated region downstream of the DNA sequence coding for the non-secreted protein. Such regions can stabilize the RNA transcript of the expression system and thus increases the yield of desired protein from the expression system. Among the 3′ untranslated regions useful in the constructs for use in the invention are sequences that provide a poly A signal. Such sequences may be derived, e.g., from the SV40 small t antigen, the casein 3′ untranslated region or other 3′ untranslated sequences well known in the art. In one aspect, the 3′ untranslated region is derived from a milk specific protein. The length of the 3′ untranslated region is not critical but the stabilizing effect of its poly A transcript appears important in stabilizing the RNA of the expression sequence.

Optionally, the construct can include a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of a gene preferentially expressed in mammary epithelial cells. For example, the N-terminal coding region can correspond to the promoter used, e.g., a goat β-casein N-terminal coding region.

The construct can be prepared using methods known in the art. The construct can be prepared as part of a larger plasmid. Such preparation allows the cloning and selection of the correct constructions in an efficient manner. The construct can be located between convenient restriction sites on the plasmid so that they can be easily isolated from the remaining plasmid sequences for incorporation into the desired mammal.

Heterologous Target Proteins

Transgenic sequences encoding heterologous target proteins can be introduced into the germline of a non-human mammal or can be transfected into a cell line as described above. The protein can be a complex or multimeric protein, e.g., a homo- or heteromultimer, e.g., proteins which naturally occur as homo- or heteromultimers, e.g., homo- or hetero-dimers, trimers or tetramers. The protein can be a protein which is processed by removal, e.g., cleavage, of N-terminus, C-terminus or internal fragments. Even complex proteins can be expressed in active form. Protein encoding sequences which can be introduced into the genome of mammal, e.g., bovines or goats, include a serum protein, a milk protein, a glycosylated or a non-glycosylated protein. The protein may be human or non-human in origin. The heterologous protein may be a potential therapeutic or pharmaceutical agent such as, but not limited to: alpha-1 proteinase inhibitor, alpha-1 antitrypsine, alkaline phosphatase, angiogenin, antithrombin III, any of the blood clotting factors including Factor VIII, Factor IX, and Factor X, bone matrix protein (e.g., BMP 1-15), chitinase, erythropoietin, extracellular superoxide dismutase, fibrinogen, glucocerebrosidase, glutamate decarboxylase, human growth factor, human serum albumin, immunoglobulin, insulin, myelin basic protein, proinsulin, prolactin, soluble CD4 or a component or complex thereof, lactoferrin, lactoglobulin, lysozyme, lactalbumin, transforming growth factor (TGF), e.g., TGF-β, tissue plasminogen activator or a variant thereof.

Nucleotide sequence information is available for several of the genes encoding the heterologous proteins listed above, in at least one, and often in several organisms. See e.g., Long et al. (1984) Biochem. 23(21):4828-4837 (aplha-1 antitrypsin); Mitchell et al. (1986) Prot. Natl. Acad. Sci USA 83:7182-7186 (alkaline phosphatase); Schneider et al. (1988) EMBO J. 7(13):4151-4156 (angiogenin); Bock et al. (1988) Biochem. 27(16):6171-6178 (antithrombin III); Olds et al. (1991) Br. J. Haematol. 78(3):408-413 (antithrombin III); Lin et al. (1985) Proc. Natl. Acad. Sci. USA 82(22):7580-7584 (erythropoeitin); U.S. Pat. No. 5,614,184 (erythropoietin); Horowitz et al. (1989) Genomics 4(1):87-96 (glucocerebrosidase); Kelly et al. (1992) Ann. Hum. Genet. 56(3):255-265 (glutamte decarboxylase); U.S. Pat. No. 5,707,828 (human serum albumin); U.S. Pat. No. 5,652,352 (human serum albumin); Lawn et al. (1981) Nucleic Acid Res. 9(22):6103-6114 (human serum albumin); Kamholz et al. (1986) Prot. Natl. Acad. Sci. USA 83(13):4962-4966 (myelin basic protein); Hiraoka et al. (1991) Mol. Cell Endocrinol. 75(1):71-80 (prolactin); U.S. Pat. No. 5,571,896 (lactoferrin); Pennica et al. (1983) Nature 301(5897):214-221 (tissue plasminogen activator); Sarafanov et al. (1995) Mol. Biol. 29:161-165, the contents of which are incorporated herein by reference.

Multivalent Binding Polypeptides

A multivalent binding polypeptide fusion protein can be prepared with standard recombinant DNA techniques using a nucleic acid molecule encoding the fusion protein. A nucleotide sequence encoding a fusion protein can be synthesized by standard DNA synthesis methods.

Methods of Determining If An Inactive Protein Can Be Secreted

Use of a Tissue Culture Assay

A construct can be engineered to express an inactive protein using the mammalian tissue culture transient expression system. For example, a gene construct can be ligated into a vector such as pcDNAII1 or pCEP4. The transfection can be carried out using standard techniques and representative samples of both supernatant and cell pellet can be obtained. Since the tissue culture system is relatively fast, the characteristics of the inactive protein can quickly be determined.

In Vivo Assays

Once it has been established that an inactive protein can be secreted and that an active protein, e.g., BMP, can then be obtained using, for example, the tissue culture system, a construct which expresses an inactive protein can be placed into the cloning site of the mammary gland expression system such as a system which includes goat beta casein. These constructs can be used to generate transgenic mice. This allows for testing the expression of the protein into milk. In addition, the health of the mammary gland and the animal can be monitored.

Methods of Determining Expression Levels

Western Blot Analysis

Using the assays described above, enough material is obtained to test expression levels using Western blots. The Western should be sensitive enough to pick up the signal for the wild type protein being secreted. If the level of expression is, for example, 10 mg/L then the level of protein in the assay would be 10 ng/ul. By running 10 ul on in each well, 100 ng should be seen on the blot.

In addition, a monoclonal that is specific for active molecules can be used to determine expression levels of an active protein. Such antibodies can be used to monitor the relative folding efficiency of the secreted proteins. Having a monoclonal specific for the active protein would be helpful to determine if an active protein is obtained after enzymatic treatment of an inactive protein.

Methods of Determining the Bioactivity of a Protein

The bioactivity of the expressed proteins can be monitored by the following methods. Since the proteins are inactive and then activated, testing of bioactivity of a protein can be performed. The bioactivity can be measured in a cell-based assay or in an in vivo model.

Production of an Inactive Protein By Modifying a Site

A protein can be transgenically produced in an inactive form by modifying a site needed for activation of the protein, e.g., a cleavage site. For example, several proteins require cleavage of a pre- and/or pro-region of the protein in order to obtain the protein in active form. Such proteins include the TGF-β family of molecules which are activated by cleavage during secretion. The cleavage site of such molecules can be modified such that cleavage does not occur during secretion. For example, TGF-β was produced in which the KKRK cleavage site was replaced with the chymosin 10 amino acid recognition sequence. The protein was secreted unclipped and then processed in the test tube with the chymosin enzyme to yield the activated protein. A similar series of experiments have been done with proinsulin. Preferably, enzymatic cleavages are used that can function on the already folded molecules and which have the ability to follow the activity of the protein.

An activation site of BMP-2 can also be modified such that cleavage does not occur during secretion of BMP-2 and it is secreted in its pro form. By replacing the normal cleavage site with amino acids of another recognition sequence (e.g., a recognition sequence which is not cleaved by an endogenous protease), the pro-form can be cleaved following purification. An activation site of BMP-2 has the following sequence: RKRLK. This sequence can be modified to provide a different cleavage site which is not cleaved during secretion of BMP-2, but can be cleaved after secretion using a proteolytic enzyme which is not normally expressed in the specific tissue or product.

Several exogenous proteolytic sites can be tried to obtain an inactive protein. These include sites for cleavage by acid, cyanogen bromide, Factor X, or chymosin. By analyzing the protein sequence, the potential sites can be designed. Various versions of a protein such as BMP with new cleavage sites can be constructed and run them through the tissue culture system. In addition, the protein can be tested to determine whether it is secreted in an inactive pro-form. Both culture supernatant and cell pellets can be tested for the levels of the protein. The pro-forms can also be tested to show that they have no biological activity.

If the inactive protein is expressed in the mammary gland, it is preferable that the additional cleavage site is not cleaved by serum proteases since the milk has serum proteins in it at a low level.

Preferably, the additional site is cleaved such that the protein includes less than 20, 10, 5, 4, 3, 2, 1, or no extraneous amino acid residues. For example, if an active BMP protein having extra amino acids on its N-terminus is desired, a site must be designed so that only the BMP sequence is present following cleavage.

The success of the cleavage step can be monitored by Western analysis. Preferably, an antibody capable or recognizing the activated protein such as active BMP is used. Once the protein is cleaved, an assay can be done for bioactivity. It will be important to run wild-type active protein through the whole system to insure that there is no inactivation is taking place. Also, testing the unprocessed protein for activity can be done. In addition, if it is found that the protein can be secreted into tissue culture as an inactive pro form that can then be activated, the constructs can then be tested in the mouse milk system. The activation of the protein can also be tested when it is mixed with mouse and goat milk.

Transgenic mice can be used to test the ability to secrete a protein in proform. For example, modified BMP-2 DNA can be ligated into a goat beta casein expression vector. The DNA can be used to generate transgenic mice that should produce the protein in their milk. Moreover, control mice can be used which express wild-type BMP-2. Milk can be obtained from representative lines, as well as biopsies for Northern analysis. The milk and tissue samples can be tested for expression of the BMP-2 proteins by Western analysis. Specifically, production of the active protein in milk can be tested using the appropriate cleavage enzymes. The activation can be monitored as was done with the tissue culture expression experiments.

Earlier attempts at producing BMP-2 in the mammary gland in active form resulted in the inhibition of mammary gland development. Thus, the levels and effects of producing the wild type as well as the novel forms of the protein can be monitored for effect on the mammary tissue.

Production of an Inactive Protein By Co-Expression of the Protein and a Binding Protein

A protein can be transgenically produced in an inactive form by co expression of the protein with a binding protein which binds to and inactivates the protein. For example, a protein and binding protein can be a receptor and ligand, or fragments or either. The binding protein can also be an antibody.

In one aspect, a protein can be expressed as a fusion protein with the binding protein. For example, BMP protein can be expressed as a fusion protein with its own receptor. The assumption is that the receptor will bind to BMP and prevent it from interacting with the receptor in the mammary gland or in the animal. The receptor can be linked to the BMP through a cleavable linker. Once the inactive version is produced, the linker can be cleaved, allowing the receptor and BMP to dissociate and thereby activating the BMP molecule. In one aspect, the cleavable linker is not recognized by an endogenous processing enzyme which naturally occurs in the tissue or product from the transgenic animal, but is recognized by processing enzymes in a subject who is administered the inactive polypeptide. Thus, the inactive polypeptide can be administered to a subject, e.g., a human, such that processing enzymes present in the subject can cleave the binding polypeptide from the target polypeptide thereby activating the target polypeptide.

A binding protein/protein fusions can be tested for expression, for example, in COS cells. Expression can be tested by analysis with monoclonal antibody directed to either the protein or the binding protein, e.g., either BMP or its receptor. The fusion protein can also be tested for activity.

In addition, to obtain an active protein such as active BMP, the fusion protein can be cleaved with the appropriate enzyme and cleavage can be monitored by Western blot. The expectation is that the fusion protein will be inactive until cleaved by the protease.

A fusion protein which includes a protein and a binding protein can also be tested for its expression in milk. The fusion construct can be ligated, for example, into the beta casein expression vector. The vector can then be used to generate transgenic mice. These mice can be tested for their ability to express the protein. In addition, the animals can be tested for the effect of the protein on the mammary gland and the animal in general.

It is also possible to express the protein along with an antibody that is capable of binding and inactivating the protein. For example, BMP and an antibody against BMP can be used to produce inactive BMP. An antibody of interest can be expressed in COS cells along with the protein such as BMP. The effect of the antibody on BMP activity can then be monitored.

An active protein such as active BMP can be obtained following expression with an antibody using, for example, Protein A to selectively bind the antibody portion of the protein/antibody complex.

The binding protein can further include at least one amino acid which is exogenous to the binding protein. For example, a BMP receptor can further include an exogenous amino acid sequence which is useful for purification of the binding protein and/or the binding protein/protein complex. For example, the additional amino acid sequence can be use to bind to a preselected ligand such as a 6X HIS ligand, a cellulose binding domain ligand, or a maltose binding domain ligand.

Various binding proteins can be tested for their ability to bind and inhibit a protein.

All patents and references cited herein are incorporated in their entirety by reference.

Other embodiments are within the following claims.

1-4. Cancel

5. A method of obtaining a target polypeptide having a bindable epitope from a product, the method comprising:
contacting a product which comprises a target polypeptide having a bindable epitope with a transgenically produced multivalent binding polypeptide, wherein the transgenically produced multivalent binding polypeptide comprises a first binding moiety which specifically binds the bindable epitope of the target polypeptide and a second binding moiety which specifically binds a matrix, to thereby provide a reaction mixture;
contacting the reaction mixture with a matrix which specifically binds the second binding moiety of the multivalent binding polypeptide;
removing reaction mixture which does not bind to the matrix, to thereby obtain the target polypeptide from the product; and
wherein the reaction mixture is substantially fluid.

6-11. cancel

12. A method of obtaining a target polypeptide having a bindable epitope from the milk of a first non-human transgenic mammal, the method comprising:
contacting milk which comprises a target polypeptide having a bindable epitope with a transgenically produced multivalent binding polypeptide, wherein the multivalent binding polypeptide comprises a first binding moiety which specifically binds the bindable epitope of the target polypeptide and a second binding moiety which specifically binds a matrix, to thereby provide a reaction mixture;
contacting the reaction mixture with a matrix which specifically binds the second binding moiety of the multivalent binding polypeptide;
removing reaction mixture which does not bind to the matrix, to thereby obtain the target polypeptide from the milk;
wherein the reaction mixture is substantially fluid; and,
wherein the transgenically produced multivalent binding polypeptide is produced in milk from a second non-human transgenic mammal.

13-30. Cancel.

31. The method of claim 5, wherein said multivalent polypeptide is used in an ELISA format.

32. The method of claim 6, wherein said target polypeptide is purified from the reaction mixture to a composition that is more than 90% pure.

33. The method of claim 5, wherein said reaction mixture is semi-solid

34. The method of claim 12, wherein said multivalent polypeptide is used in an ELISA format.

35. The method of claim 12, wherein said target polypeptide is purified from the reaction mixture to a composition that is more than 90% pure.

36. The method of claim 12, wherein said reaction mixture is semi-solid.
